(19) United States
(12) Patent Application Publication
(10) Pub. No.: US 2019/0009083 A1
(43) Pub. Date: Jan. 10, 2019

(54) Self-Powered Bone Growth Stimulator

(71) Applicant: Northeastern University, Boston, MA (US)

(72) Inventors: Thomas J. WEBSTER, Barrington, RI (US); Haridas KUMARAKURU, Quincy, MA (US)

(21) Appl. No.: 15/747,876

(22) PCT Filed: Jul. 28, 2016
(86) PCT No.: PCT/US16/44467

§ 371 Date: Jan. 26, 2018

Related U.S. Application Data
(60) Provisional application No. 62/198,014, filed on Jul. 28, 2015.

(51) Int. Cl. A61N 1/32; A61F 2/44; A61N 1/02; A61N 1/36; A61N 1/20; H01L 41/113

(52) U.S. Cl. CPC A61N 1/326; A61F 2/44; A61N 1/025; B82Y 5/00; A61N 1/205; H01L 41/113; A61N 1/36071

(57) ABSTRACT

Devices, systems, and methods for therapies involving the application of an electrical signal within the body of a subject involve the use of an implanted piezoelectric nanogenerator to provide a self-generated electrical signal without the use of batteries. The electrical signal stimulates healing of a tissue, such as bone, or provides pain relief by inhibiting neuronal pain signals. An external signal generator induces mechanical stress in an implanted piezoelectric nanomaterial, which produces the electrical signal.

### BACKGROUND

A large number of people suffer from neck and back pain due to arthritis and degenerative diseases. Approximately 432,000 spinal fusions are performed in the United States annually. In addition, a significant number of nonunion fractures in long bones occur, which cause high physical morbidity and loss of quality of life. While the body has its own natural healing process, it sometimes needs enhancement to function more effectively. Preclinical and clinical results show a promising therapeutic role for the use of electrical stimulators in bone healing, which operate by applying a stabilized electric current to the site of fracture or spinal fusion.

There are several disadvantages to the present use of electrical stimulators to promote bone growth, including limited battery life (6-8 months for DC-based stimulators) and the need for a additional surgery to replace the battery or remove the implanted device at the end of therapy. Thus, there is a need for improved devices that avoid these disadvantages.

### SUMMARY OF THE INVENTION

The invention provides a semi-invasive, cost effective, biocompatible, and substantially biodegradable bone growth stimulation system that is self-powered by a nanogenerator. The core device of this “fit and forget” system is a nanogenerator unit that is durable, highly sensitive, and implantable. The nanogenerator device utilizes ZnO nanowires or another piezoelectric material that actively produces piezoelectricity for use in various healing therapies, and particularly for the healing of bone fractures and surgically-induced bone fusions. Because the nanogenerator unit is largely or entirely biodegradable, it avoids the need for second or subsequent surgeries to replace the battery of the generator or to remove the device from the patient's body, although it can be removed at any time if the need arises.

The system is “semi-invasive” because it includes both implanted and external devices. The internal part of the system includes a self-powered nanogenerator device which produces electrical power that is fed through implanted wires to a pair of electrodes implanted at the site of a fracture or bone fusion, for example. The external part of the system includes a signal generator which can be worn on a belt at the waist, for example, where it overlays the implanted nanogenerator. The signal generator produces a mild mechanical pressure according to a pre-programmed or user selectable sequence, which is sensed by the implanted nanogenerator device, causing it to produce a DC electrical current at the site of bone repair which enhances and accelerates the repair process.

One aspect of the invention is a nanogenerator device. The device includes a substrate and a layer of piezoelectric material disposed on a surface of the substrate. The nanogenerator device is suitable for implantation in the body of a living subject and generates a current within the body of the subject in response to mechanical stress on the device.

Another aspect of the invention is a system for promoting bone growth or repair in a subject in need thereof. The system includes the nanogenerator device described above, a stimulator device capable of inducing mechanical stress in the piezoelectric material of the nanogenerator device while the stimulator device is mounted outside the body of the subject and the nanogenerator device is implanted in the body of the subject, and a pair of electrodes electrically coupled by wires to the nanogenerator device.

Yet another aspect of the invention is a method of promoting bone growth or repair in a subject in need thereof. The method includes the steps of: (a) providing the system described above; (b) implanting the nanogenerator device, pair of electrodes, and wires of the system in the body of the subject, wherein the electrodes are disposed near a site of bone growth or repair and the nanogenerator device is implanted in a location suitable for mechanostimulation by the stimulator device; (c) mounting the stimulator device of the system at an external surface of the body of the subject, whereby the stimulator device overlays the nanogenerator device; and (d) inducing mechanical stress in the piezoelectric material of the nanogenerator device using the stimulator device.

The invention can also be summarized through the following list of embodiments.

1. A nanogenerator device comprising a substrate and a layer of piezoelectric material disposed on a surface of the substrate, wherein the nanogenerator device is suitable for implantation in the body of a living subject and generates a current within the body of the subject in response to mechanical stress on the device.
2. The nanogenerator device of embodiment 1, further comprising a top layer covering the layer of piezoelectric material.
3. The nanogenerator device of embodiment 2, wherein the substrate and top layer both comprise an electrically conductive material.
4. The nanogenerator device of any of the previous embodiments, wherein the piezoelectric material is in the form of nanowires, nanorods, or nanotubes.
5. The nanogenerator device of any of the previous embodiments, wherein the piezoelectric material comprises zinc oxide nanowires.
6. The nanogenerator device of any of the previous embodiments, wherein the substrate comprises anodized titanium.
7. The nanogenerator device of any of the previous embodiments, further comprising a housing surrounding the substrate and piezoelectric material.
8. The nanogenerator device of embodiment 7, wherein the housing comprises a biodegradable material
9. The nanogenerator device of embodiment 7, further comprising two electrodes disposed on an external surface of said housing.
10. The nanogenerator device of any of the previous embodiments, further comprising two conductive leads for delivering a generated current to electrodes.
11. A system for promoting bone growth or repair in a subject in need thereof, the system comprising:

the nanogenerator device of any of the previous embodiments;

a stimulator device capable of inducing mechanical stress in the piezoelectric material of the nanogenerator device while the stimulator device is mounted outside the body of the subject and the nanogenerator device is implanted in the body of the subject; and

a pair of electrodes electrically coupled by wires to the nanogenerator device.

12. The system of embodiment 11, further comprising a belt or strap for mounting the stimulator device on an external surface of the body of the subject.
13. The system of embodiment 11 or embodiment 12, wherein the stimulator device comprises a vibration or ultrasound generator.
14. The system of embodiment 11, wherein the stimulator device comprises a programmable processor, a memory, and a display.
15. The system of any of embodiments 11-14, wherein the stimulator device further comprises a wireless transceiver.
16. The system of any of embodiments 11-15, wherein the nanogenerator device, pair of electrodes, and wires are implanted in the body of the subject.
17. A method of promoting bone growth or repair in a subject in need thereof, the method comprising the steps of:

(a) providing the system of any of embodiments 11-16;

(b) implanting the nanogenerator device, pair of electrodes, and wires of the system in the body of the subject, wherein the electrodes are disposed near a site of bone growth or repair and the nanogenerator device is implanted in a location suitable for mechanostimulation by the stimulator device;

(c) mounting the stimulator device of the system at an external surface of the body of the subject, whereby the stimulator device overlays the nanogenerator device; and

(d) inducing mechanical stress in the piezoelectric material of the nanogenerator device using the stimulator device.

18. The method of embodiment 17, wherein the site of bone growth or repair is a simple or compound bone fracture.
19. The method of embodiment 17 or embodiment 18, wherein the site of bone growth or repair is a spinal fusion.
20. The method of any of embodiments 17-19, wherein mechanical stress is induced in step (d) through the generation of vibration or ultrasound by the stimulator device.
21. The method of any of embodiments 17-20, wherein mechanical stress is induced in step (d) with the use of a programmed sequence of stimulation provided by the stimulator device.
22. The method of any of embodiments 17-21, further comprising administering one or more pharmaceutical or biotherapeutic agents that promote bone growth or remodeling.
23. The method of embodiment 22, wherein the one or more pharmaceutical or biotherapeutic agents are selected from the group consisting of bone morphogenic proteins, insulin-like growth factors, dexamethasone, fibroblast growth factor, bisphosphonates, ascorbic acid, and vitamin D.
24. The method of any of embodiments 17-23, further comprising monitoring bone growth or repair using X-rays, magnetic resonance imaging, or computed tomography.

### BRIEF DESCRIPTION OF THE DRAWINGS

FIG. 1 shows a schematic illustration of a system for promoting bone growth at a fracture through the use of a piezoelectric nanogenerator device.

FIGS. 2A-2C show schematic representations of embodiments of a piezoelectric nanogenerator device according to the invention. Dimensions are not to scale.

FIGS. 3A and 3B show scanning electron micrographs of ZnO nanowires grown on an anodized titanium substrate.

FIG. 4 shows the results of a cell proliferation study of human osteoblasts grown on plain titanium substrates (left bar of each cluster), on anodized titanium substrates containing titania nanotubes (middle bar of each cluster), and on ZnO nanowires grown on titania nanotubes with the application of mechanical force to the substrate (right bar of each cluster). The left hand cluster shows results for cells grown on plain titanium substrates; the middle cluster shows results for cells grown on titania nanotubes; and the right hand cluster shows results for cells grown on ZnO nanowires. The vertical axis shows the number of cells per well.

### DETAILED DESCRIPTION OF THE INVENTION

The invention provides devices, systems, and methods for therapies involving the application of a direct current (DC) electrical signal within the body of a subject. A key aspect of the technology is the use of an implanted piezoelectric nanogenerator to provide the DC signal, which stimulates healing of a tissue or provides pain relief by inhibiting neuronal pain signals. In a preferred embodiment, an internally generated DC signal is provided by the nanogenerator and used to promote healing of a bone fracture or a surgically-induced bone fusion.

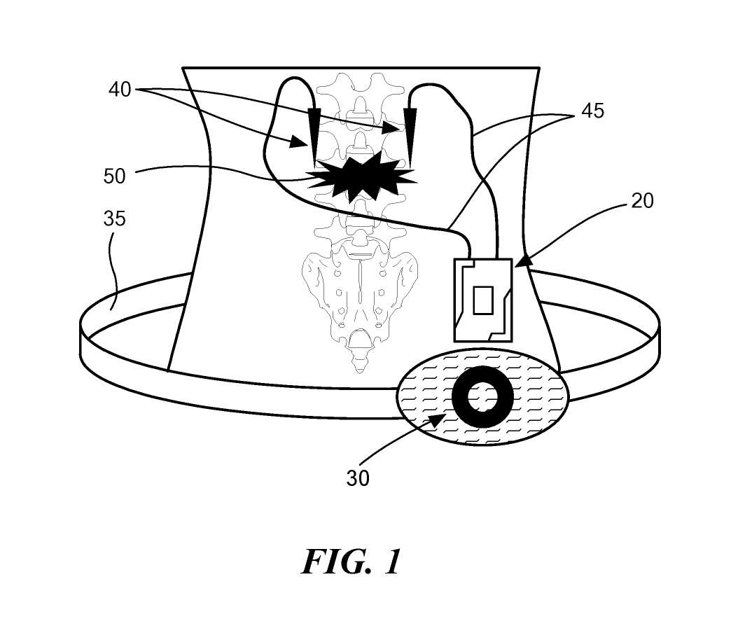

FIG. 1 depicts an embodiment of a bone growth stimulation system according to the invention. System 10 includes implanted nanogenerator device 20, which is connected via implanted electrical leads 45 to implanted electrodes 40, which are located at a site of intended bone growth, such as spinal fusion 50. External signal generator 30 is worn at the waist on belt 35, and overlays the implanted nanogenerator, to which it imparts mechanical force according to a program or user settings. The signal generator can include an electromechanical transducer, such as a piston, diaphragm, vibrator, or ultrasound generator for the generation of mechanical force. Mechanical force also can be generated passively, by simply tightening belt 35 so as to impart a force against the nanogenerator through the patient's body.

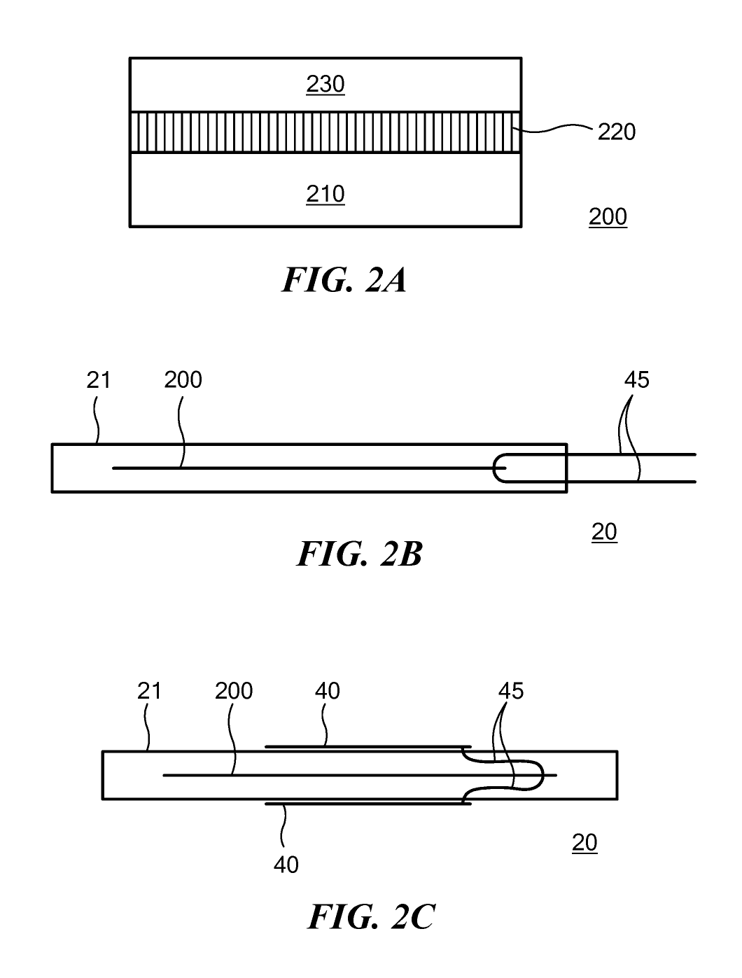

FIGS. 2A-2C depict embodiments of nanogenerator device 20. In the embodiment of FIG. 2A, piezoelectric nanowires 220 are aligned in a layer disposed on a surface of substrate 210. Optional top layer 230 covers the nanowire layer and protects it from damage, but also aids in the distribution of mechanical force over the nanowire layer. Preferably, the top layer includes or consists of a conductive metal such as gold, silver, copper, aluminum, or chromium, and also can be used to collect electrical charges separated in the piezoelectric material by applied mechanical stress and conduct a direct current to one of the electrodes. Similarly, if the substrate is electrically conductive, it can also assist in charge collection, and can conduct direct current to the other electrode to complete the circuit. In the embodiment of FIG. 2B, nanogenerator chip 200, containing the substrate, piezoelectric nanowires, and optional top layer, is mounted in housing 21, and electrical leads or wires 45 are connected to the chip and directed out through the housing where they can be led through the subject's body to the electrodes at the site of treatment. The embodiment shown in FIG. 2C is wireless, and utilizes electrodes 40 mounted on housing 21; wires 45 are internal to the nanogenerator device housing, and connect the nanogenerator chip to the electrodes. The configuration, position, and surface area of the electrodes as well as the shape, size, and configuration of the nanogenerator housing will determine the distribution of the generated electric field and current flow, and can be freely selected according to the needs of the application.

The nanogenerator device is implanted within the subject's body at a location near the intended site of healing or at a site remote therefrom. The nanogenerator contains a piezoelectric material which is preferably in the form of nanowires, and which can be a crystalline material having a cylindrical or other extended form and having an aspect ratio of at least 3 (i.e., length to width ratio of 3:1 or greater), or at least 5, 7, 10, or greater. The dimensions of the nanowires can be, for example a width of about 5, 10, 15, 20, 25, 30, 40, 50, 60, 70, 80, 90 100, 150, 200, 250, 300, 400, or 500 nm, or up to about 999 nm, and a length of about 10, 20, 30, 40, 50, 60, 70, 80, 90, 100, 150, 200, 250, 300, 400, 500, 700, 1000, 1500, 2000, 2500, 3000, 3500, 4000, 5000, 6000, 7000, 8000, 9000, or 10000 nm or more. In a preferred embodiment, the nanowires have a size of about 10 nm wide and about 100 nm long.

The piezoelectric nanowires can be arranged within the nanogenerator so as to allow them to sense a mechanical force applied from outside the body through a stimulator device. Preferably, the nanowires are deposited or grown on a substrate, and their orientation is ordered or highly ordered, with their longitudinal axis perpendicular to the substrate (i.e., vertically aligned with respect to the substrate). Optionally, a top layer, such as a gold layer, is deposited onto the nanowires at the face of the nanowire layer oriented away from the substrate. The top layer can aid in absorbing mechanical forces and transmitting or focusing them onto the nanowires. Such mechanical forces can be constant over a period of time, or slowly or rapidly varying, such as induced by an external vibrator or ultrasound transmitter in the stimulator device. The use of vibration or ultrasound can produce output electricity from the nanogenerator in a pulsed format.

ZnO is a preferred piezoelectric material for the nanogenerator device due to its unique semiconducting and piezoelectric properties. These properties can be optimized for use in the invention by selecting from among different nano-architectures. As a semiconductor, ZnO is a cheap and earth abundant raw material having a direct band gap of 3.37 eV, a large exciton binding energy (60 meV), excellent chemical and thermal stability as well as biocompatibility, and high radiation tolerance. Piezoelectricity is a self-generated form of electricity produced by charges that accumulate on parallel faces of a piezoelectric material, such as a ZnO crystal, when the material is subjected to an external pressure via mechanical squeezing or stretching. ZnO can be grown in various nanostructure forms, including nanorods and nanowires, and on a variety of substrates using laboratory friendly and cost effective techniques. Moreover, ZnO is a biocompatible and biodegradable material which has been used in many biomedical applications, including biosensors, anti-bacterial agents, cancer cell diagnostic and therapeutic agents, and drug delivery vehicles. Other piezoelectric materials that can be used in the nanogenerator device include carbon nanotubes, barium sulfate, and lead titanate. Methods are known for growing piezoelectric crystalline and other materials in the form of nanowires, nanorods, or nanotubes. Such methods include, but are not limited to, metal organic chemical vapor deposition (MOCVD), molecular beam epitaxy (MBE) and pulsed laser deposition (PLD).

The substrate material can be any material that provides a rigid mechanical support for the piezoelectric nanowires. In certain embodiments the substrate is electrically conductive and aids in transmitting separated charge from the piezoelectric material to the electrodes or into surrounding tissue. A preferred substrate material is anodized titanium (i.e., a titanium sheet having a surface coating of titania nanotubes), but other materials such as stainless steel, CoCr, titanium alloys, alumina, and tantalum also can be used. Substrates are preferred that include nanotubes or nanopores on their surface, as these can serve to both nucleate nanowire or nanorod growth and also to establish the orientation (especially the vertical alignment) and distribution of the nanowires or nanorods on the substrate.

The nanogenerator device preferably includes a housing in which the nanogenerator substrate, nanowires, and optional top surface layer are mounted. The housing and other components of the nanogenerator device preferably utilize biocompatible and biodegradable polymers where possible. Examples of suitable biodgradable polymers include poly-lactic acid, poly-glycolic acid, and polyesters. Optionally, the housing outer surface is coated with an antimicrobial coating material. The housing may include openings for two electrode leads that transmit the generated current from the nanogenerator to the electrodes. The size of the nanogenerator and its housing are kept as small as possible, consistent with the amount of power that needs to be generated. For example, the nanogenerator housing can have a largest external dimension in the range from about 1 mm to about 10 cm, and is preferably in the range from about 1 cm to about 5 cm.

A bone growth stimulator system of the invention can include or consist of one or more nanogenerators which are embedded near the site of bone growth and repair and emit a direct current into the surrounding tissue in response to mechanical forces imposed on the nanogenerator(s) by ordinary movements of the subject's body. Alternatively, the system can also include an externally mounted stimulator device that provides programmed mechanical stimulation to the nanogenerator. When an external stimulator is used, it is preferably worn on the body, either strapped or taped in place, or worn on a waist belt or harness, and overlying one or more nanogenerator devices implanted below the stimulator at a depth of about 10-15 mm. The depth is preferably kept to a minimum so as to permit transmission of mechanical forces between the stimulator and the generator.

The bone growth stimulation system of the present invention utilizes wires to connect the current generator to the electrodes, which achieves greater efficiency than wireless systems. Preferably the generator produces a constant direct current (DC) which is delivered through implanted wires to implanted electrodes situated on either side of a fracture or site of intended bone fusion. The bone healing and regeneration process requires a steady and uniform current and electric field as established by a DC power supply. For example, a nanogenerator device of the present invention produces a DC voltage in the range from about 10 mV to about 1000 mV, or from about 10 mV to about 500 mV, or from about 10 mV to about 100 mV, or from about 30 mV to about 60 mV. Other systems, such as spinal cord stimulation systems, for example, disturb pain signals using AC signals and can employ noninvasive wireless technology. The present technology can also produce and utilize alternating currents for controlling cell responses, such as reducing pain, controlling drug release from polymers through electrical degradation of the polymer, decreasing bacterial infection, increasing nerve regeneration, promoting vascular tissue growth, and controlling stem cell differentiation.

The invention also contemplates methods of using the devices and systems disclosed herein to promote, enhance, and/or accelerate the growth, repair, and/or remodeling of bone fractures and bone fusions. Such methods utilize a nanogenerator device either alone or as part of a bone growth and repair system. The nanogenerator device, with its pair of electrodes and wires connecting the electrodes to the nanogenerator, are surgically implanted into the body of the subject. The subject is a mammal, preferably a human. A preferred location for the nanogenerator is in the abdomen, just under the skin; the device can be placed by laparotomy or laparoscopy. The wires leading from the nanogenerator device are routed under the skin to a pair of electrodes disposed near a desired site of bone growth or repair. Following surgical implantation of the internal parts of the system, an external stimulator device is positioned at an external surface of the body of the subject, at a position that allows the stimulator device to overlie the implanted nanogenerator device. Finally, mechanical stress is induced by the external stimulator in the piezoelectric material of the implanted nanogenerator device.

Mechanical stress can be induced in a number of possible ways, including by compression achieved through tightening of a belt on which the stimulator is mounted, and generation of mechanical vibration or ultrasound by a transducer in the stimulator, appropriately aimed at the nanogenerator device. In certain embodiments, the mechanical stress is induced with the help of a programmed sequence of stimulation provided by the stimulator device. The device can be programmed, for example, to provide a suitable stimulus amplitude, frequency, interval, time of onset, or a combination of such stimulus features. Through the use of a wireless transceiver, the function of the stimulator can be remotely monitored or its programming altered. A display on the stimulator can be used to indicate stimulator status, function, program number, program progress, or to assist the user in programming the device or selecting a program.

The method can also include the administration of one or more pharmaceutical or biotherapeutic agents that promote bone growth or remodeling. Such agents can work synergistically with the electrical stimulation provided by the system. The one or more pharmaceutical or biotherapeutic agents can be selected from, for example, bone morphogenic proteins (including BMP-7 or OP-1™), insulin-like growth factors, dexamethasone, fibroblast growth factor, bisphosphonates, ascorbic acid, and vitamin D.

The method can also include monitoring bone growth or repair using X-rays, magnetic resonance imaging, or computed tomography.

### EXAMPLES

### Example 1. Preparation of Titania Nanotubes

Titanium foils (99.5% Ti, 0.25 mm thick, annealed) and platinum meshes were purchased from Alfa Aesar. Other chemicals were purchased from Sigma-Aldrich or Fisher Scientific. Ti foils were cut into 2.5 cm×2.5 cm squares and were cleaned with acetone, 70% ethanol, and deionized water (Milli-Q water) separately, each for 15 minutes. Then, the cleaned Ti foils were etched for 1 minute with a solution containing nitric acid solution (1.5% by weight) and hydrofluoric acid (1.5% by weight) to remove the naturally occurring oxide layer.

The Ti foils were then anodized using a two-electrode configuration, with a Pt mesh serving as the cathode and a Ti foil serving as the anode. One side of each of the Ti and Pt electrodes was immersed in an electrolyte solution consisting of 1% HF, while the other side of each electrode was connected to a DC power supply through copper wires. Anodization proceeded for 10 minutes at 20 V, during which titania nanotubes were grown on the side of the Ti foil contacting the electrolyte solution. After anodization, the Ti substrates were rinsed immediately with large amounts of deionized water and dried in an oven at 100° C. for 30 minutes.

### Example 2. Synthesis of ZnO Nanowires on Titania Nanotube Substrates

Zinc oxide nanowires were synthesized on anodized titanium substrates of Example 1 by two different methods. For either method, the substrate first was ultrasonically cleaned in acetone followed by ethanol and de-ionized water for five minutes in each solvent at room temperature, followed by drying under a nitrogen stream for 5 minutes at room temperature.

Method 1

Commercial zinc oxide nanoparticles (Nanophase Technologies Inc.) were seeded onto a substrate surface containing titania nanotubes via spin coating of a well agitated ethanolic solution containing 10 mM of zinc acetate dihydrate and polyvinylpyrrolidone. Following the spin coating process, the seeded substrate was annealed in air within a furnace at 200° C. for 120 minutes.

Method 2

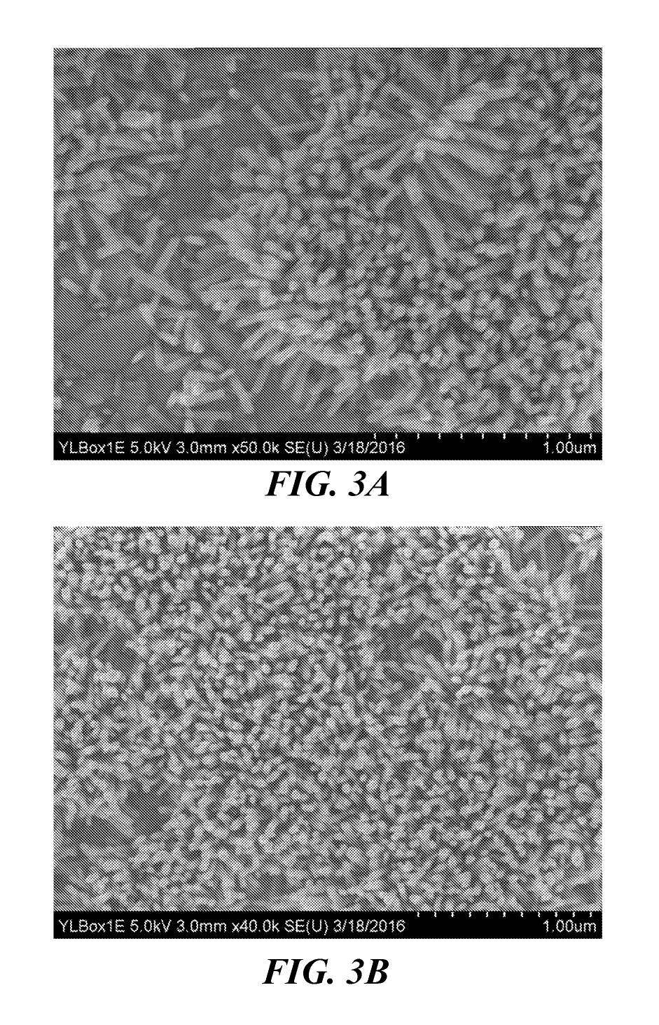

Zinc carbonate nanoparticles were precipitated onto a titania substrate containing titania nanotubes by combining 1M zinc nitrate and 1M ammonium carbonate in an aqueous solution in which the substrate was immersed at room temperature. Zinc carbonate was then allowed to precipitate onto and within the Ti nanotubes for 12 hours at room temperature. The next day, the Zn-seeded Ti substrate was then attached to a microscope slide using Teflon tape and placed in an aqueous solution containing 50 mM zinc nitrate hexahydrate and 50 mM hexamethylenetetramine (hexamine) held at 85° C. under reflux for 90 minutes, during which zinc oxide nanowires were produced on the substrate. Following removal from the solution, the substrate was rinsed with deionized water and then dried under a nitrogen steam. ZnO nanowires produced by this method are shown in FIGS. 3A and 3B.

Following deposition of ZnO nanowires by either method, the substrates were sterilized via UV light before used in cell culture experiments.

### Example 3. Effect of ZnO Nanowire-Generated Potential on Osteoblast Growth

Osteoblasts were grown on substrates (either pure Ti, anodized Ti with nanotubes, or anodized Ti with ZnO nanowires grown out of the titania nanotubes) for up to 5 days with mechanical stimulation. Mechanical stimulation was applied to the piezoelectric ZnO nanowires via an ADMET mechanical testing system to generate an electrical potential, whose influence on cell proliferation was tested.

Human osteoblasts obtained from PromoCell, Heidelberg, Germany, were used at population numbers less than ten for all cell experiments. Cells were cultured in Eagle's Minimum Essential Medium (EMEM; ATCC) supplemented with 10% fetal bovine serum (FBS; Sigma-Aldrich) and 1% penicillin/streptomycin (P/S; Gibco) or Dulbecco's Modified Eagle Medium (DMEM; ATCC) supplemented with 10% FBS and 1% P/S. The cells were seeded onto the ZnO nanowire/TiO₂ nanotube substrates at a density of 5000 cells/cm² and were allowed to grow for 1, 3, or 5 days in a 37° C. incubator in a humidified, 5% CO₂ atmosphere. At the end of the incubation time period, the substrates were washed twice with PBS and were transferred to fresh 24-well tissue culture plates. Next, 150 μL of MTT dye solution (Promega MTT Cell Proliferation Assay) was added to each well, and the plates were cultured for another 4 hours. Following the incubation, 1 ml of MTT stop solution (Promega) was added to each well, and the plates were incubated overnight. A plate reader (Molecular Devices, SpectraMax M3, 570 nm) was used to determine cell density.

Mechanical force was applied to all the anodized Ti substrates, including those containing ZnO nanowires, with bone cells using a 10 lb load cell in an ADMET Biotense Perfusion Bioreactor and software (ADMET, Inc.). Substrates were cut into 2.5 cm squares and secured within the grips of the device. The Ti substrate with ZnO nanowires and osteoblasts was coated with a collagen casing (mimicking that of tissue which would be found above the material if implanted) and subjected to compression and tension using grips that moved accordingly at a rate of 0.1 mm/min for 5 minutes at which time the force was released and the same process was repeated. The entire construct was bathed in cell culture medium. The mechanical compressive force caused the generation of an electrical potential which influenced osteoblast function.

Following incubation of the osteoblasts under application of mechanical force for the indicated time periods, the substrates were thoroughly rinsed with deionized water and then dried at room temperature. Samples were characterized by scanning electron microscopy using a Hitachi S-4800 microscope. A palladium layer was created on the samples using a sputter coater (Cressington Sputter Coater 208HR) to make them conductive.

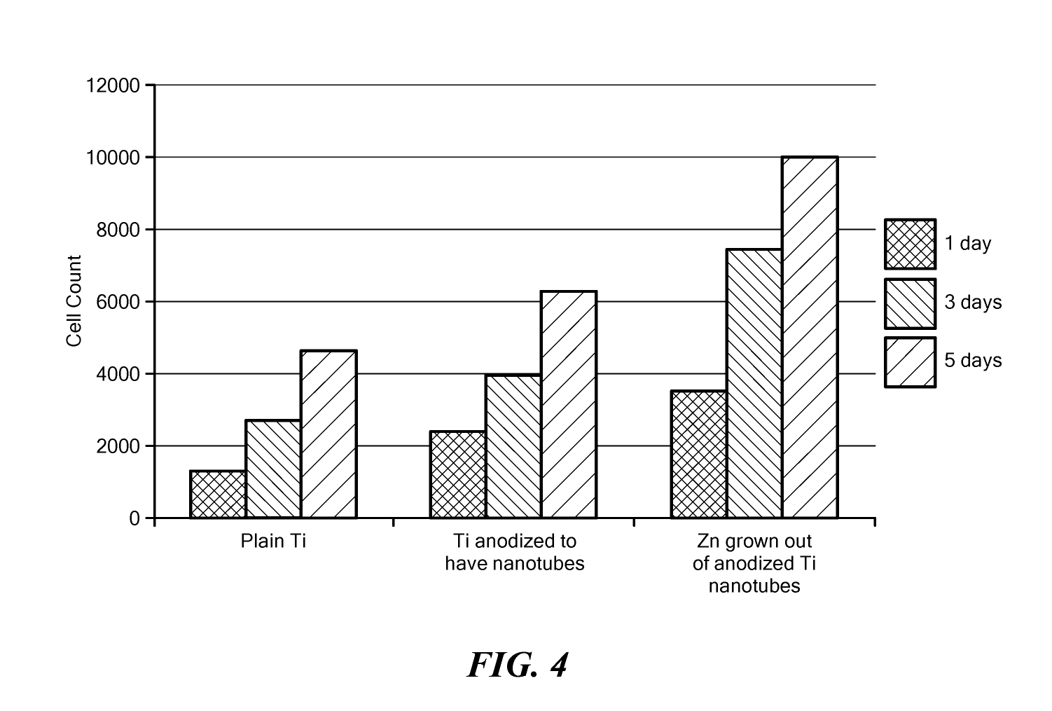

The results of the cell proliferation study are shown in FIG. 4. At each condition, cell number increased progressively from day 1 to day 3 to day 5. The presence of nanotubes on the substrate stimulated cell growth compared to a plain Ti substrate. A substantial further increase in cell proliferation was observed in the presence of ZnO nanowires in conjunction with the application of mechanical force to generate an electrical potential across the nanowires. The results were consistent with promotion of bone growth in response to electrical signals produced by mechanical stimulation of ZnO nanowires.

This application embodiments the priority of U.S. Provisional Application No. 62/198,014 filed 28 Jul. 2015 and entitled “Fit and Forget Electrical Stimulators”, the whole of which is hereby incorporated by reference.

As used herein, “consisting essentially of” allows the inclusion of materials or steps that do not materially affect the basic and novel characteristics of the embodiment. Any recitation herein of the term “comprising”, particularly in a description of components of a composition or in a description of elements of a device, can be exchanged with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

What is claimed is:

1. A nanogenerator device comprising a substrate and a layer of piezoelectric material disposed on a surface of the substrate, wherein the nanogenerator device is suitable for implantation in the body of a living subject and generates a current within the body of the subject in response to mechanical stress on the device.

2. The nanogenerator device of claim 1, further comprising a top layer covering the layer of piezoelectric material.

3. The nanogenerator device of claim 2, wherein the substrate and top layer both comprise an electrically conductive material.

4. The nanogenerator device of claim 1, wherein the piezoelectric material is in the form of nanowires, nanorods, or nanotubes.

5. The nanogenerator device of claim 4, wherein the piezoelectric material comprises zinc oxide nanowires.

6. The nanogenerator device of claim 1, wherein the substrate comprises anodized titanium.

7. The nanogenerator device of claim 1, further comprising a housing surrounding the substrate and piezoelectric material.

8. The nanogenerator device of claim 7, wherein the housing comprises a biodegradable material

9. The nanogenerator device of claim 7, further comprising two electrodes disposed on an external surface of said housing.

10. The nanogenerator device of claim 1, further comprising two conductive leads for delivering a generated current to electrodes.

11. A system for promoting bone growth or repair in a subject in need thereof, the system comprising:
the nanogenerator device of claim 1;
a stimulator device capable of inducing mechanical stress in the piezoelectric material of the nanogenerator device while the stimulator device is mounted outside the body of the subject and the nanogenerator device is implanted in the body of the subject; and
a pair of electrodes electrically coupled by wires to the nanogenerator device.

12. The system of claim 11, further comprising a belt or strap for mounting the stimulator device on an external surface of the body of the subject.

13. The system of claim 11, wherein the stimulator device comprises a vibration or ultrasound generator.

14. The system of claim 11, wherein the stimulator device comprises a programmable processor, a memory, and a display.

15. The system of claim 14, wherein the stimulator device further comprises a wireless transceiver.

16. The system of claim 11, wherein the nanogenerator device, pair of electrodes, and wires are implanted in the body of the subject.

17. A method of promoting bone growth or repair in a subject in need thereof, the method comprising the steps of:
(a) providing the system of claim 11;
(b) implanting the nanogenerator device, pair of electrodes, and wires of the system in the body of the subject, wherein the electrodes are disposed near a site of bone growth or repair and the nanogenerator device is implanted in a location suitable for mechanostimulation by the stimulator device;
(c) mounting the stimulator device of the system at an external surface of the body of the subject, whereby the stimulator device overlays the nanogenerator device; and
(d) inducing mechanical stress in the piezoelectric material of the nanogenerator device using the stimulator device.

18. The method of claim 17, wherein the site of bone growth or repair is a spinal fusion.

19. The method of claim 17, wherein mechanical stress is induced in step (d) through the generation of vibration or ultrasound by the stimulator device.

20. The method of claim 17, wherein mechanical stress is induced in step (d) with the use of a programmed sequence of stimulation provided by the stimulator device.

21. The method of claim 17, further comprising administering one or more pharmaceutical or biotherapeutic agents that promote bone growth or remodeling.

22. The method of claim 17, further comprising monitoring bone growth or repair using X-rays, magnetic resonance imaging, or computed tomography.
